(19) United States
(12) Patent Application Publication
(10) Pub. No.: US 2010/0312121 A1
(43) Pub. Date: Dec. 9, 2010

(54) APPARATUS FOR A NEEDLE DIRECTOR FOR AN ULTRASOUND TRANSDUCER PROBE

(76) Inventor: Zhonghui Guan, Burlingame, CA (US)

Correspondence Address: Zhonghui Guan, 2941 Arguello Dr, Burlingame, CA, 94010, US

(21) Appl. No.: 12/481,469

(22) Filed: Jun. 9, 2009

(51) Int. Cl. A61B 8/00

(52) U.S. Cl. 600/461

(57) ABSTRACT

An apparatus for a needle director for an ultrasound transducer probe includes an adapter for joining to the probe. The adapter includes a bottom residing in a first plane perpendicular to a midline of the central plane of the ultrasound transducer probe. A needle insertion site is disposed in the first plane for allowing the needle to pass below the first plane. A plate is disposed in a second plane parallel to and offset from a third plane containing the midline and the needle insertion site. The plate has a flat surface proximate the third plane for guiding an insertion of the needle along the third plane. The flat surface has a plurality of radial lines having indicators of distances along the midline and a curved line intersecting the plurality of radial lines for aligning with a marker on the needle to indicate that a tip of an inserted needle has intersected the midline.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to medical equipment. More particularly, the invention relates to needle directors for ultrasound-guided procedures.

### BACKGROUND OF THE INVENTION

Ultrasound is widely used in medical practice. It provides two-dimensional images of the inside structures of human bodies. With the help of ultrasound images, physicians can insert needles into human bodies to reach specific places for many medical purposes. For example, if a tumor is detected by the ultrasound, an intervention radiologist can insert a needle into the tumor to obtain tumor tissue for pathological examination. During an ultrasound-guided procedure, the ultrasound probe must be covered by a sterile cover to keep the procedure field sterile.

However, to successfully perform an ultrasound guided biopsy and other ultrasound-guided procedures, both target tissue (i.e., a tumor) and the needle, especially the needle tip, must be visualized simultaneously. There are two techniques that are widely used in clinical practice to visualize the target tissue and the needle in ultrasound-guided procedures, the “in plane” technique and the “off plane” technique.

Because the image of an ultrasound is only two-dimensional, showing the central plane of the ultrasound transducer probe, the only way to visualize the entire needle is to keep the entire needle in the central plane of the ultrasound probe. This is the basic principle of the “in plane” technique. In clinical practice, the ultrasound probe is held by a physician in one hand and the needle is inserted with the other hand. It requires a lot of clinical training and high skill to coordinate both hands to keep the entire needle and the target in the central plane of the transducer probe simultaneously, and it can be very time consuming. Many ultrasound-guided procedures, such as an ultrasound-guided peripheral nerve block by an anesthesiologist, require injecting or sampling at multiple sites. Therefore it is very important that the needle is free to be inserted at different directions while it is still kept within the central plane when the “in plane” technique is applied. In addition, different ultrasound-guided procedures require different sizes of needles. For example, small needles are required in an ultrasound-guided peripheral nerve block, whereas larger needles are usually used in fluid aspiration or tissue biopsy.

Many devices have been designed to keep the needle in the central plane of the ultrasound transducer probe to make sure that the needle is visualized by the ultrasound probe. Most of these devices have complicated components, and the majority of these devices have fixed insertion angles. As a result, the clinical application of these devices has been greatly limited. There are some existing devices that allow the direction of needle insertion to be changed relatively freely during the procedure. These devices are designed to have either a multi-joint arm or a slot. However, the devices with a multi-joint arm are too cumbersome for clinical use. For devices with a slot, it is usually very difficult to insert needles of different sizes through the single size of the slot, and therefore it is very difficult to make one such device that fits all needles.

With the “in plane” technique, quite often the needle shaft is in the central plane and therefore is visualized by the ultrasound, whereas the needle tip can be off the plane and therefore is not visualized, even with the help from a device that is designed to keep the needle in plane, especially when the target is deep inside patient's body. The partial visualization of the needle often gives the physician a false judgment about the location of the needle tip, which can be dangerous because the unseen needle tip can damage other tissue. A solution that can estimate the location of the needle tip even if it is slightly off the central plane and not detected by the ultrasound is very desirable. However, so far there is no device that can estimate the needle tip location other than direct visualization by ultrasound.

The “off plane” technique, which is much easier to perform than the “in plane” technique, does not require the entire needle to be kept on the central plane of the transducer probe. Instead, the needle is inserted some distance away from the central plane, and it is inserted toward the central plane. The needle is visualized only when it passes the central plane. However, only the part of the needle that is in the central plane is visualized. In other words, if the needle tip is not directly in the central plane, it will not be visualized, and there is no way to know the exact location of the needle tip. Therefore, with the “off plane” technique, it is absolutely crucial to stop the needle tip at the central plane of the transducer probe. If the needle is too shallow for the needle tip to reach the central plane, the needle tip is not visualized. If the needle is too deep and the needle tip passes the central plane, the needle tip is not visualized either. Without knowing the exact location of the needle tip, the ultrasound-guided procedure can be dangerous and ineffective. In general, the “off plane” technique is considered to be associated with higher risks. So far there is no existing device that can help to locate the needle tip with the “off plane” technique.

Another problem of ultrasound-guided procedures is that, with both the “in plane” and the “off plane” techniques, there is no current solution for a physician to choose an accurate needle insertion direction beforehand. With the “in plane” technique, the current practice is that the needle is first inserted into patient's body. It is then visualized by ultrasound. Once the needle is visualized, it is redirected within the patient's body, multiple times if necessary, until the needle is directed toward the target. This practice, however, greatly increases the risk of the procedure and the discomfort of the patient because the needle is inserted and redirected multiple times within patient's body before it reaches its final direction. It also significantly prolongs the procedure because a lot of time is consumed in finding the right insertion direction. A solution that can determine the insertion direction before the needle is inserted into the patient is greatly desirable. With the “off plane” technique, the situation is worse because the needle tip is not be visualized until it reaches the central plane. As a result, it is often very difficult to know if the needle is heading toward the target before the needle is blindly inserted quite deep into the patient. Therefore for the “off plane” technique to be safely applied, a device is badly needed to determine the needle insertion direction before the needle is inserted into the patient.

In view of the foregoing, there is a significant need for improved techniques for directing a needle during an ultrasound-guided procedure that can determine the insertion direction of the needle before the procedure and can estimate the location of the needle tip during the procedure.

### BRIEF DESCRIPTION OF THE DRAWINGS

The present invention is illustrated by way of example, and not by way of limitation, in the figures of the accompanying drawings and in which like reference numerals refer to similar elements and in which:

FIGS. 1A, 1B and 1C illustrate an exemplary “in plane” needle director and a needle, in accordance with an embodiment of the present invention. FIG. 1A is a bottom view of the needle director. FIG. 1B is a front view of the needle director, and FIG. 1C shows the needle that is marked for use with the needle director;

FIGS. 2A and 2B illustrate an exemplary “off plane” needle director, in accordance with an embodiment of the present invention. FIG. 2A is a bottom view, and FIG. 2B is a lateral view;

FIGS. 3A and 3B illustrate an exemplary “in plane” needle director with a needle holder and a joint, in accordance with an embodiment of the present invention. FIG. 3A is a bottom view, and FIG. 3B is a front view; and

FIGS. 4A and 4B illustrate an exemplary “off plane” needle director with a needle holder and a single joint, in accordance with an embodiment of the present invention. FIG. 4A is a bottom view, and FIG. 4B is a lateral view.

Unless otherwise indicated illustrations in the figures are not necessarily drawn to scale.

### SUMMARY OF THE INVENTION

To achieve the forgoing and other objects and in accordance with the purpose of the invention, an apparatus for a needle director for an ultrasound transducer probe is presented.

In one embodiment an apparatus for a needle director for an ultrasound transducer probe is presented. The apparatus includes means for joining to the ultrasound transducer probe, means for allowing the needle to pass below the joining means, means for visualizing a whole needle or a needle tip in ultrasound image while guiding an insertion of the needle to reach a specific point of the central plane of the ultrasound transducer probe, means for indicating an insertion direction for the needle to reach a specific point of the central plane of the ultrasound transducer probe, and means for indicating an insertion distance for a tip of the needle to reach a specific point of the central plane of the ultrasound transducer probe. Other embodiments further include means for pivotally holding the needle and means for joining the holding means to the joining means. Another embodiment further includes means for maintaining a sterile field.

In another embodiment an apparatus for a needle director for an ultrasound transducer probe is presented. The apparatus includes an adapter for joining to the ultrasound transducer probe. The adapter includes a bottom residing in a first plane perpendicular to a midline of a central plane of the ultrasound transducer probe. A needle insertion site is disposed for allowing the needle to pass below the first plane. A needle used for insertion is marked with at least one marker that is certain distance from the needle tip. A plate, joined to the adapter, is disposed in a second plane parallel to and offset from a third plane containing a specific line in the central plane of the ultrasound transducer probe and the needle insertion site. The plate has a first flat surface proximate the third plane for guiding an insertion of the needle along the third plane. The first flat surface has a plurality of radial lines having indicators of distances along the specific line in the central plane. The first flat surface further includes at least one curved line intersecting the plurality of radial lines for aligning with a marker on the needle to indicate that a tip of an inserted needle has intersected the specific line in the central plane. In another embodiment the specific line in the central plane of the ultrasound transducer probe is the midline of the central plane of the ultrasound transducer probe. In another embodiment the third plane is in a central plane of the ultrasound transducer probe for an in plane insertion of the needle. In still another embodiment the third plane is perpendicular to a central plane of the ultrasound transducer probe for a typical off plane insertion of the needle. In yet another embodiment the third plane is any plane that contains the specific line in the central plane of the ultrasound transducer probe for an atypical off plane insertion of the needle. In still another embodiment the curved line that intersects the plurality of radial lines is a curved edge of the plate. Another embodiment has a sheet joined to the bottom and a sheet joined to the top for maintaining a sterile field. Another embodiment further includes a needle holder pivotally joined to the adapter for allowing the needle to pivot in the third plan. In another embodiment the needle holder further includes a needle canal. Yet another embodiment further includes a bridge for joining the needle holder to the adapter.

In another embodiment an apparatus for a needle director for an ultrasound transducer probe is presented. The apparatus includes an adapter for joining to a distal portion of the ultrasound transducer probe. The adapter includes a bottom residing in a first plane parallel to a distal end of the joined ultrasound transducer probe and perpendicular to a midline of the ultrasound transducer probe where the midline extends through a center of a central plane of the ultrasound transducer probe. A needle insertion site is disposed in the first plane for allowing the needle to pass below the first plane. A needle used for insertion is marked with at least one marker that is certain distance from the needle tip. A plate is joined to the adapter for guiding the needle. The plate is disposed in a second plane parallel to and offset from a third plane containing the midline and the needle insertion site. The plate has a flat edge positioned in the first plane and a first flat surface proximate the third plane for guiding an insertion of the needle along the third plane. The first flat surface includes a plurality of radial lines corresponding to insertion directions extending through the needle insertion site. Each of the plurality of radial lines has an indicator of a distance along the midline where a corresponding insertion direction intersects. The first flat surface further includes at least one curved line intersecting the plurality of radial lines for aligning with a marker on an inserted needle for indicating that a tip of an inserted needle has intersected the midline at a distance indicated by at least one of the indicators. In another embodiment the third plane is in a central plane of the ultrasound transducer probe for an in plane insertion of the needle. In still another embodiment the third plane is perpendicular to a central plane of the ultrasound transducer probe for a typical off plane insertion of the needle. In yet another embodiment the third plane is any plane that contains the midline in the central plane of the ultrasound transducer probe for an atypical off plane insertion of the needle. In another embodiment the curved line is a curved edge of the plate. In yet another embodiment the adapter surrounds the distal portion of the ultrasound transducer probe. Another embodiment further includes a needle holder pivotally joined to the adapter for holding the needle and allowing the needle to pivot in the third plan about the insertion site. In another embodiment the needle holder further includes a needle canal. Yet another embodiment further includes a bridge for joining the needle holder to the adapter. Still another embodiment further includes a sheet joined to the bottom for maintaining a sterile field. Still another embodiment further includes a sheet jointed on the top to cover the proximal portion of the ultrasound transducer probe for maintaining a bigger sterile filed.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention will now be described in detail with reference to embodiments thereof as illustrated in the accompanying drawings.

Preferred embodiments of the present invention facilitate ultrasound-guided procedures, which can be time consuming and sometimes can be dangerous to patients. Preferred embodiments of the present invention provide devices that help to determine needle insertion directions, to visualize needles by ultrasound transducer probes, to estimate needle tip locations, to allow needle insertion directions to be changed freely, and to keep the procedure field sterile. Some devices according to preferred embodiments can be used with any sized needles and thus are universal needle directors. Preferred embodiments make ultrasound-guided procedures much safer, faster, easier, and much more tolerable for patients. Compared with existing needle guides, preferred embodiments are simple and have independent, simple methods to assess the needle insertion direction and the needle tip location. Physicians who are involved in ultrasound-guided procedures would likely employ preferred embodiments of the present invention. In fact, there is a great need for this type of device in medical practice.

The ultrasound image is a two-dimensional image detected at the central plane of an ultrasound transducer probe. Needle directors according to preferred embodiments are devices that are attached to the transducer probe to facilitate needle insertion. The “central plane” refers to the central plane of the ultrasound transducer probe, the plane in which ultrasound image is available. The “midline” refers the center of the ultrasound image and therefore the midline of the ultrasound transducer probe. In medical practice, the target is usually kept in the center of the ultrasound image, and therefore at the midline of the transducer probe. The depth of the needle target, or the distance between the target and the ultrasound transducer probe, can be reported by most ultrasound machines.

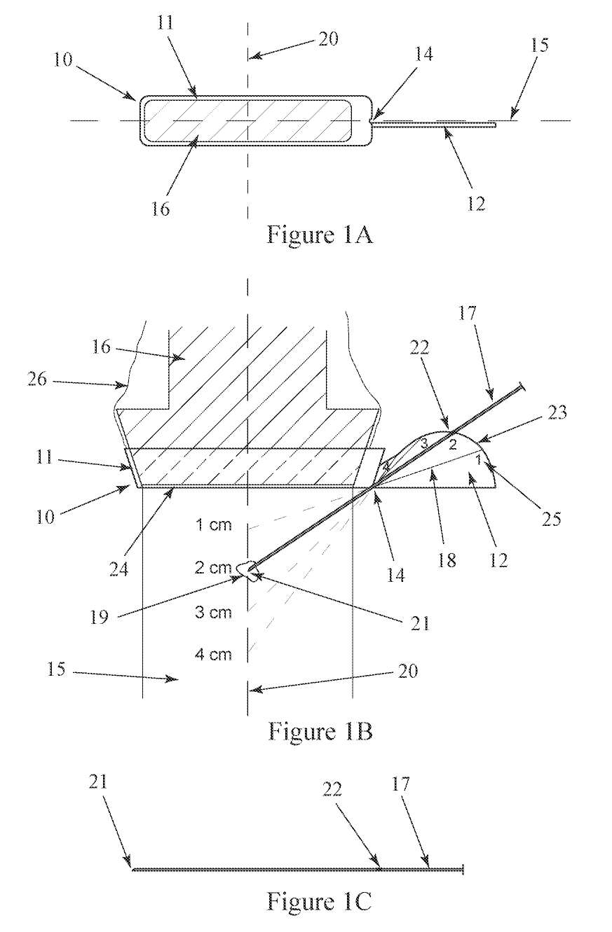

FIGS. 1A, 1B and 1C illustrate an exemplary “in plane” needle director 10 and a needle 17, in accordance with an embodiment of the present invention. FIG. 1A is a bottom view of needle director 10. FIG. 1B is a front view of needle director 10, and FIG. 1C shows needle 17 that is marked for use with needle director 10. In the present embodiment, needle director 10 comprises an adaptor 11, a wing 12, and a needle insertion site 14. Adaptor 11 is designed in a way that the distal portion of an ultrasound transducer probe 16 can be fitted into adaptor 11 perfectly. Needle insertion site 14 is directly in the central plane 15 of ultrasound transducer probe 16, which is the plane of the ultrasound image. Wing 12 is a flat plate that is parallel to but a short distance, for example, without limitation, 0.5 mm, away from central plane 15 and is attached to adaptor 11 beside needle insertion site 14. Therefore, if needle 17 is inserted at insertion site 14 and advanced in full contact with wing 12, needle 17 remains in central plane 15 and thus is visualized by the ultrasound image. In alternate embodiments the wing may be positioned closer to or further from the central plane depending on various factors. In the present embodiment, wing 12 comprises multiple lines 18 that are drawn from insertion site 14 indicating the direction of needle insertion needed to reach a target 19 if target 19 is at the center of the ultrasound image, or at a midline 20, and is a certain distance away from ultrasound transducer probe 16. Lines 18 are labeled by numbers 25 that indicate the depth of target 19. For example, without limitation, in the present embodiment, the line labeled by the number 1 indicates the needle insertion direction when target 19 is 1 cm deep and is at the center of the ultrasound image on midline 20, whereas the line labeled by the number 3 indicates the needle insertion direction when target 19 is 3 cm deep and is at the center of the ultrasound image on midline 20. If the depth of target 19 is between 2 cm and 3 cm, needle 17 should be inserted in a direction that is between the line labeled with a 2 and the line labeled with a 3. Alternate embodiments may have multiple needle insertion sites, and different insertion directions may use different insertion sites. In other alternate embodiments the wing may comprise various different markings or guides to indicate the direction of needle insertion such as, but not limited to, dots, dashes, grooves, raised areas, etc. Furthermore, alternate embodiments may use various different means for indicating the depth of the target for example, without limitation, different units of measurement may be used, more or fewer depths may be indicated, etc.

In the present embodiment, the shape of wing 12 is designed and needle 17 is marked in a way that if needle 17 is inserted at insertion site 14 and advanced in full contact with wing 12, the tip 21 of needle 17 should hit midline 20, which is the center of the image, when a marker 22 on needle 17 reaches an edge 23 of wing 12, no matter what direction needle 17 is inserted. In the present embodiment, marker 22 is 6 cm from tip 21; however, the distance from the tip to the marker in alternate embodiments may vary depending on factors such as, but not limited to, the size of the wing, the size of the ultrasound transducer probe, etc. In another alternate embodiment, a similarly shaped curve or multiple curves can be drawn on the wing to serve the same function as the curve of edge 23. In yet other alternative embodiments the needle may have multiple markers for each insertion direction. Other alternate embodiments may be implemented without markings on the needle or an indication on the wing showing the depth of the needle.

In the present embodiment, a sheet 24 at the distal end of adaptor 11 separates ultrasound transducer probe 16 from the patient. Another sheet 26 can be added at the proximal end of adaptor 11 to completely cover the proximal portion of ultrasound transducer probe 16. The entirety of needle director 10 can be made as a single sterile piece to be used as a disposable device, thus an extra sterile cover sheath for the ultrasound transducer probe is not needed to keep the procedure field sterile. In an alternate embodiment the sheet at the distal and/or the proximal ends of the adaptor may be removable so that the device may be reused by attaching a new, sterile sheet to the device for each procedure. The sheet may be made removable using various different means including, but not limited to, adhesive, clips, screws, hook and loop material, etc.

Compared to existing needle guides, needle director 10 is very simple and is much more informative. Wing 12, which is parallel to yet a short distance away from central plane 15 of ultrasound transducer probe 16, helps to secure needle 17 in central plane 15. Therefore, needle 17 is visualized in the ultrasound image provided needle 17 is inserted while in full contact with wing 12. The needle insertion direction can be changed freely as long as needle 17 remains fully in contact with wing 12 to remain visualized by the ultrasound image. The single wing design of the present embodiment enables any size of needle to be used with needle director 10. Wing 12 can also provide information about the needle insertion direction and the location of needle tip 21 if needle 17 is inserted at needle insertion site 14. None of the existing needle guides can provide either insertion direction or needle tip location. Although one existing needle guide does mark insertion angles, in practice it is nearly impossible to determine if needle should be inserted at a certain angle, such as, but not limited to, a 30-degree angle or at a 45-degree angle. Although it is ideal to insert needle 17 at needle insertion site 14, needle 17 can be inserted at any other places in the central plane, provided the needle is inserted while in full contact with wing 12. However, if needle 17 is not inserted at needle insertion site 14, wing 12 may not provide accurate information about insertion direction or the location of needle tip 21.

Referring to FIG. 1B, in typical use of the present embodiment, a user inserts the distal end of ultrasound transducer probe 16 into needle director 10. The whole set (ultrasound transducer probe 16 and needle director 10) is put on the patient at specific location so that target 19 is in the center of the ultrasound image. In order to determine the insertion direction, the user obtains the depth of target 19, which is typically reported by the ultrasound machine. In this example target 19 is 2 cm deep. Therefore, the user aligns needle 17 with the line labeled with a 2 to achieve the correct insertion direction to reach target 19, when target 19 is located on midline 20. The user then inserts needle 17 into needle insertion site 14 while keeping needle 17 in full contact with wing 12 along the line labeled with number 2. When marker 22 on needle 17 reaches edge 23 of wing 12, tip 21 of needle 17 should have reached target 19 with needle 17 visualized in the ultrasound image the entire time. The user may then perform the procedure on target 19, for example, without limitation, injecting medication, taking a tissue sample, etc.

In an alternate embodiment, the wing of the needle director may be directly aligned with the insertion site and the central plane of the ultrasound transducer probe, and may comprise channels extending through the wing from the insertion site into which the needle is inserted. These channels may also be marked with various lines, numbers, letters or other markers that provide information such as, but not limited to, needle insertion direction, needle tip location, target depth, etc. Completely inserting the needle into a channel generally ensures that the needle remains aligned with the central plane. However, these embodiments may limit the sizes of needles that may be used with a particular needle director, and the insertion direction may not be changed freely. Needle directors according to these embodiments may be made in specific sizes to correspond to needles of specific size.

A basic embodiment of an “in plane” needle director comprises a flat plate that is parallel to yet, without limitation, 0 to a few millimeters away from the central plane of the ultrasound transducer probe. In some embodiments the flat plate can be designed as a part of the transducer probe itself, as a part of variously shaped adaptors that are attached to the transducer probe or as a separate component that can be attached to transducer probes or adaptors. In some of these basic embodiments of an “in plane” needle director, the flat plates may comprise various lines, curves, numbers, letters or other markers that provide information such as, but not limited to, needle insertion direction, needle tip location, target depth, etc. The needle director may also be shaped in certain ways to provide similar information.

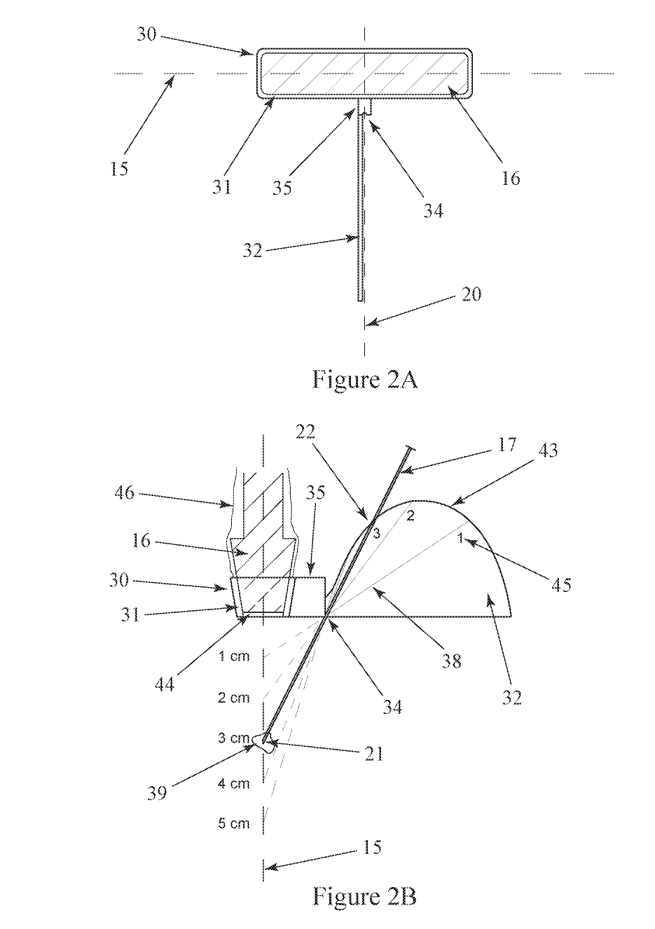

FIGS. 2A and 2B illustrate an exemplary “off plane” needle director 30, in accordance with an embodiment of the present invention. FIG. 2A is a bottom view, and FIG. 2B is a lateral view. In the present embodiment, needle director 30 comprises an adaptor 31, a wing 32, a needle insertion site 34, and a bridge 35 that links wing 32 and needle insertion site 34 to adaptor 31. Adaptor 31 is designed in a way that the distal portion of an ultrasound transducer probe 16 can be fitted into it perfectly. Needle insertion site 34 is designed some distance away, for example, without limitation, 5 mm, from ultrasound transducer probe 16 and is in the midline 20 of ultrasound transducer probe 16. Since ultrasound transducer probe 16 is narrower in the “off plane” direction than in the “in plane” direction, bridge 35 creates the space between insertion site 34 and ultrasound transducer probe 16 to enable needle 17 to be inserted at a less vertical angle. Alternative embodiments may not contain the bridge. In other alternate embodiments the insertion site and the wing may be positioned closer to or further from the ultrasound transducer probe.

In the present embodiment, wing 32 is a flat plate that is adjacent to needle insertion site 34 and is perpendicular to the central plane 15 of ultrasound transducer probe 16. Wing 32 is a short distance, for example, without limitation, 0.5 mm, away from midline 20. Therefore, needle 17 remains along midline 20 if needle 17 is inserted at insertion site 34 and if needle 17 is advanced in full contact with wing 32. As a result, when needle 17 reaches central plane 15 of ultrasound transducer probe 16 and is visualized by the ultrasound image, needle 17 is at the center of the ultrasound image. In alternate embodiments the wing may be positioned closer to or further from the midline depending on various factors. In the present embodiment, wing 32 comprises multiple lines 38 that are drawn from insertion site 34 indicating the direction of needle insertion needed to reach a target 39 if target 39 is at the center of the ultrasound image and is a certain distance away from ultrasound transducer probe 16. In the present embodiment, numbers 45 indicate the depth of target 39. For example, without limitation, the line labeled by the number 1 indicates the needle insertion direction when target 39 is 1 cm deep and is at the center of the ultrasound image, whereas the line labeled by the number 3 indicates the needle insertion direction when target 39 is 3 cm deep and is at the center of the ultrasound image. If the depth of target 39 is between 2 cm and 3 cm, needle 17 should be inserted in a direction that is between the line labeled with a 2 and the line labeled with a 3. Alternate embodiments may have multiple needle insertion sites, and different insertion directions may use different insertion sites. In other alternate embodiments the wing may comprise various different markings and guides to indicate the direction of needle insertion such as, but not limited to, dots, dashes, grooves, raised areas, etc. Furthermore, alternate embodiments may use various different means for indicating the depth of the target for example, without limitation, different units of measurement may be used, more or fewer depths may be indicated, etc.

In the present embodiment, the shape of wing 32 is designed and needle 17 is marked in a way that if needle 17 is inserted at needle insertion site 34 and is advanced in full contact with wing 32, the tip 21 of needle 17 should hit central plane 15 of ultrasound transducer probe 16 and be visualized at the center of the ultrasound image when a specific marker 22 reaches an edge 43 of wing 32, regardless of the needle insertion direction. In the present embodiment, marker 22 is 6 cm from tip 21 on needle 17; however, the needles in alternate embodiments may be marked in different locations depending on factors such as, but not limited to, the size of the wing, the size of the ultrasound transducer probe, etc. In some alternate embodiments, a similarly shaped curve or curves can be drawn on the wing to serve the same function as edge 43. In yet other alternative embodiments the needle may have multiple markers for each insertion direction. Other alternate embodiments may be implemented without markings on the needle or an indication on the wing showing the depth of the needle.

In the present embodiment, a sheet 44 at the distal end of adaptor 31 separates ultrasound transducer probe 16 from the patient. Another sheet 46 can be added at the proximal end of adaptor 31 to completely cover the proximal portion of ultrasound transducer probe 16. The entirety of needle director 30 can be made as a single sterile piece to be used as a disposable device, thus an extra sterile cover sheath for the ultrasound transducer probe is not needed to keep the procedure field sterile. In an alternate embodiment the sheet at the distal and/or proximal ends of the adaptor may be removable so that the device may be reused by attaching a new, sterile sheet to the device for each procedure. The sheet may be made removable using various different means including, but not limited to, adhesive, clips, screws, hook and loop material, etc.

Referring to FIG. 2B, in typical use of the present embodiment, a user inserts the distal end of ultrasound transducer probe 16 into needle director 30. The whole set (ultrasound transducer probe 16 and needle director 30) is put on the patient at specific location so that target 39 is in the center of the ultrasound image. In order to determine the insertion direction, the user obtains the depth of target 39, which is typically reported by the ultrasound machine. In this example target 39 is 3 cm deep. Therefore, the user aligns needle 17 with the line labeled with a 3 to achieve the correct insertion direction to reach target 39, when target 39 is located on central plane 15 and midline 20. The user then inserts needle 17 into needle insertion site 34 while keeping needle 17 in full contact with wing 32 along the line labeled with number 3. When marker 22 on needle 17 reaches edge 43 of wing 32, tip 21 of needle 17 should reach target 39 and central plane 15 and should be visualized in the ultrasound image. The user may then perform the procedure on target 39, for example, without limitation, injecting medication, taking a tissue sample, etc.

In an alternate embodiment, the wing of the needle director may be directly aligned with the insertion site and the midline of the ultrasound transducer probe, and may comprise channels extending through the wing from the insertion site into which the needle is inserted. These channels may also be marked with various lines, numbers, letters or other markers that provide information such as, but not limited to, needle insertion direction, needle tip location, target depth, etc. Completely inserting the needle into the channels generally ensures that the needle remains aligned with the midline. However, these embodiments may limit the sizes of needles that may be used with a particular needle director, and the insertion direction may not be changed freely. Needle directors according to these embodiments may be made in specific sizes to correspond to needles of specific size.

A basic embodiment of an “off plane” needle director comprises a flat plate that is perpendicular to the central plane of the ultrasound transducer probe. This flat plate may be placed at the midline of the ultrasound transducer probe or in other places, for example, without limitation, 0.5 millimeter to a few centimeters away for the midline. The flat plate may be designed as a part of the ultrasound transducer probe itself, as a part of variously shaped adaptors that are attached to the ultrasound transducer probe, or as a separate component that can be attached to ultrasound transducer probes or adaptors. In some of these basic embodiments, various lines, curves, numbers, letters or other markers can be added on the flat plate to provide further information, such as, but not limited to, needle insertion direction, needle tip location, target depth, etc. The plate may also be shaped in certain ways to provide similar information.

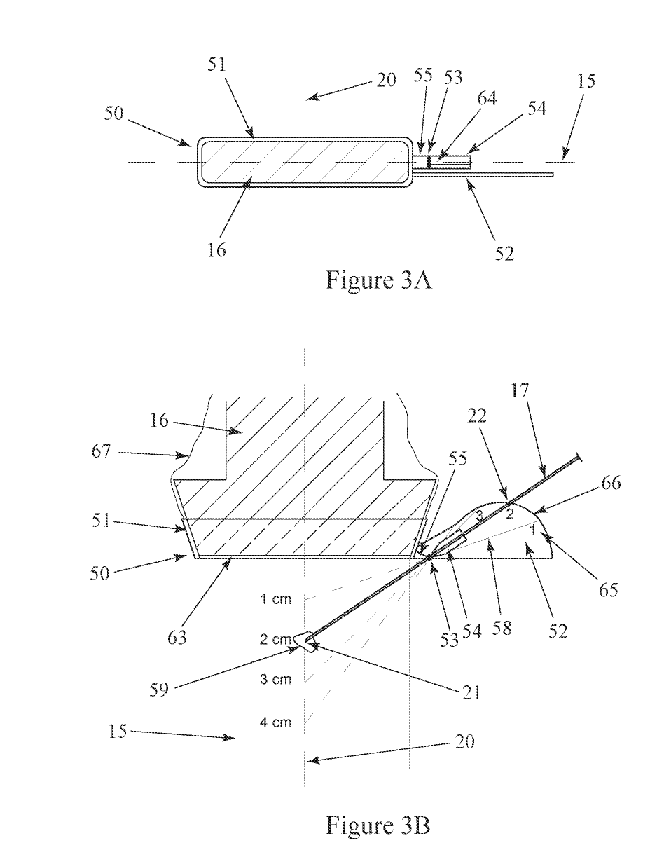

FIGS. 3A and 3B illustrate an exemplary “in plane” needle director 50 with a needle holder 54 and a single joint 53, in accordance with an embodiment of the present invention. FIG. 3A is a bottom view, and FIG. 3B is a front view. In the present embodiment, needle director 50 comprises an adaptor 51, a wing 52, a needle holder 54, a joint 53, and a bridge 55 that links needle holder 54 and joint 53 to adaptor 51. Adaptor 51 is designed in a way that the distal portion of an ultrasound transducer probe 16 can be fitted into it perfectly. Joint 53 attaches needle holder 54 to adaptor 51 in a way that a needle canal 64 of needle holder 54 is in the center plane 15 of ultrasound transducer probe 16. Joint 53 is perpendicular to both central plane 15 of ultrasound transducer probe 16 and needle canal 64 so that when needle holder 54 moves around joint 53, needle canal 64, through which a needle 17 is inserted, remains in central plane 15 of ultrasound transducer probe 16. Joint 53 is at the distal end of both needle holder 54 and adaptor 51 and is in contact with the patient during use. The opening of needle canal 64, which is the needle insertion site, travels directly through the center of joint 53. Bridge 55 creates space between adaptor 51 and needle holder 54 to permit the range of motion of needle holder 54. In alternative embodiments the joint may not be at the distal end of the needle holder. Moreover, alternative embodiments may not contain the bridge.

Wing 52 is a flat plate that is parallel to central plane 15 and needle canal 64 and is attached to adaptor 51 some distance, for example, without limitation, 1 mm, away from needle holder 54. In alternate embodiments the wing may be positioned closer to or further from the needle holder. In the present embodiment, wing 52 comprises multiple lines 58 that are drawn from joint 53 indicating the direction of needle insertion needed to reach a target 59 if target 59 is at the center of the ultrasound image, or on a midline 20, and is a certain distance away from ultrasound transducer probe 16. Lines 58 are labeled by numbers 65 that indicate the depth of target 59. For example, without limitation, the line labeled by the number 1 indicates the needle insertion direction when target 59 is 1 cm deep and is at the center of the ultrasound image, whereas the line labeled by the number 3 indicates the needle insertion direction when target 59 is 3 cm deep and is at the center of the ultrasound image. If the depth of target 59 is between 2 cm and 3 cm, needle 17 should be inserted in a direction that is between the line labeled with a 2 and the line labeled with a 3. In alternate embodiments the wing may comprise various different markings to indicate the direction of needle insertion such as, but not limited to, dots, dashes, grooves, raised areas, etc. Furthermore, alternate embodiments may use various different means for indicating the depth of the target for example, without limitation, different units of measurement may be used, more or fewer depths may be indicated, etc.

In the present embodiment, the shape of wing 52 is designed and needle 17 is marked in a way that if needle 17 is inserted through needle canal 64 of needle holder 54, the tip 21 of needle 17 should hit midline 20, which is the center of the ultrasound image, when a specific marker 22 on needle 17 reaches an edge 66 of wing 52, regardless of the direction in which needle 17 is inserted. In the present embodiment, marker 22 is 6 cm from tip 21; however, the distance from the tip to the marker in alternate embodiments may vary depending on factors such as, but not limited to, the size of the wing, the size of the ultrasound transducer probe, etc. In other alternate embodiments, a similarly shaped curve or curves can be drawn on the wing to serve the same function as edge 66. In yet other alternative embodiments the needle may have multiple markers for each insertion direction. In yet other alternate embodiments, the mark on the needle may align with the proximal end of the needle holder or markers on the needle holder rather than the edge of the wing. Yet other alternate embodiments may be implemented without markings on the needle or an indication on the wing showing the depth of the needle.

In the present embodiment, a sheet 63 at the distal end of adaptor 51 separates ultrasound transducer probe 16 from the patient during the procedure. Another sheet 67 can be added at the proximal end of adaptor 11 to completely cover the proximal portion of ultrasound transducer probe 16. The entirety of needle director 50 can be made as a single sterile piece to be used as a disposable device, thus an extra sterile cover sheath for ultrasound transducer probe 16 is not needed to keep the procedure field sterile. In an alternate embodiment the sheet at the distal and/or proximal ends of the adaptor may be removable so that the device may be reused by attaching a new, sterile sheet to the device for each procedure. The sheet may be made removable using various different means including, but not limited to, adhesive, clips, screws, hook and loop material, etc.

Referring to FIG. 3B, in typical use of the present embodiment, a user inserts the distal end of ultrasound transducer probe 16 into needle director 50. The whole set (ultrasound transducer probe 16 and needle director 50) is put on the patient at specific location so that target 59 is in the center of the ultrasound image. In order to determine the insertion direction, the user obtains the depth of target 59, which is typically reported by the ultrasound machine. In this example target 59 is 2 cm deep. Therefore, the user aligns needle holder 54 with the line labeled with a 2 to achieve the correct insertion direction to reach target 59, when target 59 is located on midline 20. The user then inserts needle 17 into needle canal 64 of needle holder 54 along the line labeled with number 2. When marker 22 on needle 17 reaches edge 66 of wing 52, tip 21 of needle 17 should reach target 59 with needle 17 visualized in the ultrasound image the entire time. The user may then perform the procedure on target 59, for example, without limitation, injecting medication, taking a tissue sample, etc.

A basic embodiment of an “in plane” needle director with a needle holder comprises a needle holder with a needle canal in the central plane of the ultrasound transducer probe and a joint that is perpendicular to the central plane and the needle canal. This basic embodiment does not comprise a wing and, therefore, does not provide information about the angle of insertion or depth of the needle. This needle holder may be designed as a part of the ultrasound transducer probe itself, as a part of variously shaped adaptors that are attached to the ultrasound transducer probe, or as a separate component that can be attached to the ultrasound transducer probe or its adaptors. In an alternate embodiment, an adaptor, a wing, a bridge, and a joint may be made as a single universal piece. Onto this piece different needle holders with different sizes of needle canals may be attached. This enables needles of different sizes to be used with the present embodiment.

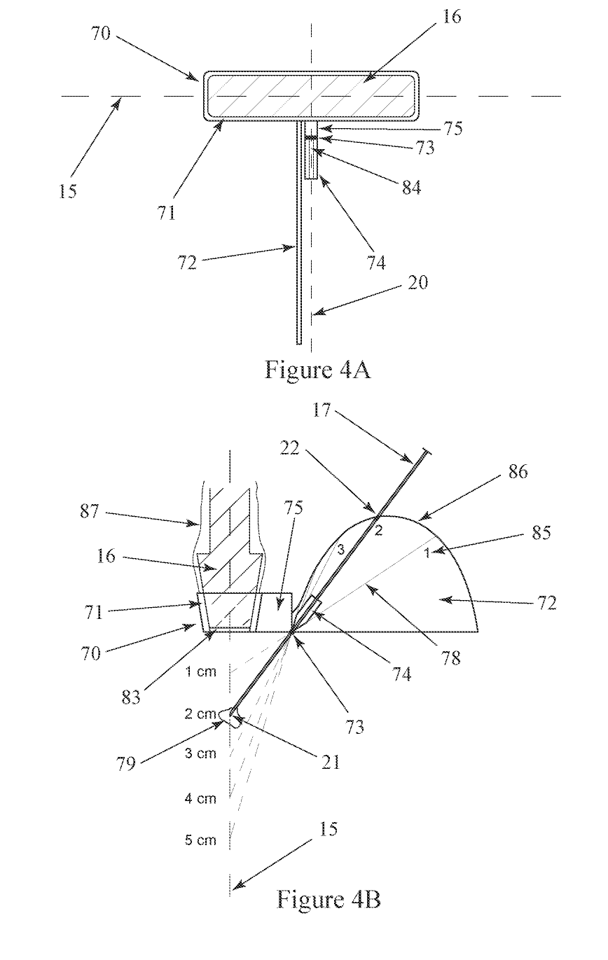

FIGS. 4A and 4B illustrate an exemplary “off plane” needle director 70 with a needle holder 74 and a single joint 73, in accordance with an embodiment of the present invention. FIG. 4A is a bottom view, and FIG. 4B is a lateral view. In the present embodiment, needle director 70 comprises an adaptor 71, a wing 72, a needle holder 74, a joint 73, and a bridge 75. An adaptor 71 is designed in a way that a distal portion of an ultrasound transducer probe 16 can be fitted into it perfectly. Bridge 75 links needle holder 74 and joint 73 to the center of the long side of adaptor 71 so that a needle canal 84 of needle holder 74 is in the center of ultrasound transducer probe 16 and therefore in the plane of a midline 20. Joint 73 is at the distal end of needle holder 74 and is parallel to a central plane 15 of ultrasound transducer probe 16 and perpendicular to needle canal 84 through which a needle 17 is inserted. As a result, the plane in which needle canal 84 moves by joint 73 remains perpendicular to central plane 15 of ultrasound transducer probe 16 and contains midline 20. The opening of needle canal 84, which is the needle insertion site, is directly through the center of joint 73. Bridge 75 creates space between adaptor 71 and needle holder 74 to enable a larger range of motion than if needle holder 74 were attached directly to adaptor 71. In alternative embodiments the joint may not be at the distal end of the needle holder. Moreover, alternative embodiments may not contain the bridge.

In the present embodiment, wing 72 is a flat plate that is parallel to the plane in which needle canal 84 moves and is perpendicular to central plane 15 of ultrasound transducer probe 16. Wing 72 is attached to adaptor 71 some distance, for example, without limitation, 1 mm, away from needle holder 74. In alternate embodiments, the wing may be attached to the adaptor closer to or further from the needle holder. In the present embodiment, wing 72 comprises multiple lines 78 that are drawn from joint 73 indicating the direction of needle insertion needed to reach a target 79 if target 79 is at the center of the ultrasound image, or on midline 20, and is a certain distance away from ultrasound transducer probe 16. Lines 78 are labeled by numbers 85 that indicate the depth of target 79. For example, without limitation, the line labeled by the number 1 indicates the needle insertion direction when target 79 is 1 cm deep and is at the center of the ultrasound image, whereas the line labeled by the number 3 indicates the needle insertion direction when target 79 is 3 cm deep and is at the center of the ultrasound image. If the depth of target 79 is between 2 cm and 3 cm, needle 17 should be inserted in a direction that is between the line labeled with a 2 and the line labeled with a 3. In alternate embodiments the wing may comprise various different markings or guides to indicate the direction of needle insertion such as, but not limited to, dots, dashes, grooves, raised areas, etc. Furthermore, alternate embodiments may use various different means for indicating the depth of the target for example, without limitation, different units of measurement may be used, more or fewer depths may be indicated, etc.

In the present embodiment, the shape of wing 72 is designed and needle 17 is marked in a way that if needle 17 is inserted through needle canal 84, the tip 21 of needle 17 should hit central plane 15 of ultrasound transducer probe 16 and be visualized by the ultrasound image when a specific marker 22 on needle 17 reaches an edge 86 of wing 72, regardless of the needle insertion direction. In the present embodiment, marker 22 is 6 cm from tip 21 on needle 17; however, the needles in alternate embodiments may be marked in different locations depending on factors such as, but not limited to, the size of the wing, the size of the ultrasound transducer probe, etc. In some alternate embodiments, a similarly shaped curve or curves can be drawn on the wing to serve the same function as edge 86. In yet other alternative embodiments the needle may have multiple markers for each insertion direction. In yet other alternate embodiments, the mark on the needle may align with the proximal end of the needle holder or markers on the needle holder rather than the edge of the wing. Other alternate embodiments may be implemented without markings on the needle or an indication on the wing showing the depth of the needle.

In the present embodiment, a sheet 83 at the distal end of adaptor 71 separates ultrasound transducer probe 16 from the patient. Another sheet 87 can be added at the proximal end of adaptor 11 to completely cover the proximal portion of ultrasound transducer probe 16. The entirety of needle director 70 may be made as a single sterile piece to be used as a disposable device, thus an extra sterile cover sheath for ultrasound transducer probe 16 is not needed to keep the procedure field sterile. In an alternate embodiment the sheet at the distal and/or proximal ends of the adaptor may be removable so that the device may be reused by attaching a new, sterile sheet to the device for each procedure. The sheet may be made removable using various different means including, but not limited to, adhesive, clips, screws, hook and loop material, etc.

Referring to FIG. 4B, in typical use of the present embodiment, a user inserts the distal end of ultrasound transducer probe 16 into needle director 70. The whole set (ultrasound transducer probe 16 and needle director 70) is put on the patient at specific location so that target 79 is in the center of the ultrasound image. In order to determine the insertion direction, the user obtains the depth of target 79, which is typically reported by the ultrasound machine. In this example target 79 is 2 cm deep. Therefore, the user aligns needle holder 74 with the line labeled with a 2 to achieve the correct insertion direction to reach target 79, when target 79 is located on central plane 15 and midline 20. The user then inserts needle 17 into needle canal 84 of needle holder 74 along the line labeled with number 2. When marker 22 on needle 17 reaches edge 86 of wing 72, tip 21 of needle 17 should reach target 79 and central plane 15 and should be visualized in the ultrasound image. The user may then perform the procedure on target 79, for example, without limitation, injecting medication, taking a tissue sample, etc.

A basic embodiment of an “off plane” needle director with a needle holder comprises a needle holder with a needle canal moving in a plane that is perpendicular to the central plane of the ultrasound transducer probe and the joint that is parallel to the central plane and perpendicular to the needle canal. The needle director can be placed at the midline of the ultrasound transducer probe or in other places such as, but not limited to, a few centimeters away from the midline. This basic embodiment does not comprise a wing and, therefore, does not provide information about the angle of insertion or depth of the needle. The needle director may be designed as a part of ultrasound transducer probe itself, as a part of variously shaped adaptors that are attached to the ultrasound transducer probe, or as a separate component that can be attached to ultrasound transducer probes or adaptors. In an alternate embodiment, an adaptor, a wing, a bridge, and a joint may be made as a single universal piece. Onto this piece different needle holders with different sizes of needle canals may be attached. This enables needles of different sizes to be used with the present embodiment.

Having fully described at least one embodiment of the present invention, other equivalent or alternative methods of providing a needle guide for ultrasound procedures according to the present invention will be apparent to those skilled in the art. The invention has been described above by way of illustration, and the specific embodiments disclosed are not intended to limit the invention to the particular forms disclosed. For example, the particular implementation of the adaptor may vary depending upon the particular type of ultrasound probe used. The ultrasound probes described in the foregoing were directed to trapezoidal implementations; however, similar techniques are to make adaptors to fit probes of various shapes and sizes such as, but not limited to, round adaptors, rectangular adaptors, etc. Furthermore, adaptors in some embodiments may be adjustable to fit multiple types of ultrasound probes. For example, without limitation, in some embodiments the adaptor may be made of material with elastic properties, such as, but not limited to, rubber, that enables the adaptor to stretch over various different probes and in other embodiments the adaptor may have an adjustable collar that enables it to be tightened onto the probe. Implementations of the present invention with varying types of adaptors are contemplated as within the scope of the present invention. The invention is thus to cover all modifications, equivalents, and alternatives falling within the spirit and scope of the following claims.

What is claimed is:

1. An apparatus for a needle director for an ultrasound transducer probe, the apparatus comprising:
means for joining to the ultrasound transducer probe;
means for allowing the needle to pass below said joining means;
means for visualizing a whole needle or a needle tip in ultrasound image while guiding an insertion of the needle to reach a specific point of the central plane of the ultrasound transducer probe;
means for indicating an insertion direction for the needle to reach a specific point of the central plane of the ultrasound transducer probe; and
means for indicating an insertion distance for a tip of the needle to reach a specific point of the central plane of the ultrasound transducer probe.

2. The apparatus as recited in claim 1, further comprising means for pivotally holding the needle.

3. The apparatus as recited in claim 2, further comprising means for joining said holding means to said joining means.

4. The apparatus as recited in claim 1, further comprising means for maintaining a sterile field.

5. An apparatus for a needle director for an ultrasound transducer probe, the apparatus comprising:
an adapter for joining to the ultrasound transducer probe, said adapter comprising a bottom residing in a first plane perpendicular to a midline of a central plane of the ultrasound transducer probe;
a needle insertion site for allowing the needle to pass below said first plane;

6. The apparatus as recited in claim 5, further comprising
A needle with at least one marker that is certain distance from the needle tip; and
a plate, joined to said adapter, disposed in a second plane parallel to and offset from a third plane containing a specific line in the central plane of the ultrasound transducer probe and said needle insertion site, said plate comprising a first flat surface proximate said third plane for guiding an insertion of the needle along said third plane, said first flat surface comprising a plurality of radial lines having indicators of distances along said specific line in the central plane, said first flat surface further comprising at least one curved line intersecting said plurality of radial lines for aligning with said marker on the needle to indicate that a tip of an inserted needle has intersected said specific line in the central plane.

7. The apparatus as recited in claim 5, wherein a specific line in the central plane of the ultrasound transducer probe is the midline of the central plane of the ultrasound transducer probe.

8. The apparatus as recited in claim 5, wherein said third plane is in a central plane of the ultrasound transducer probe for an in plane insertion of the needle.

9. The apparatus as recited in claim 5, wherein said third plane is perpendicular to a central plane of the ultrasound transducer probe for a typical off plane insertion of the needle.

10. The apparatus as recited in claim 5, wherein said third plane is any plane that contains the specific line in the central plane of the ultrasound transducer probe other than the planes recited in claim 7 and 8 for an atypical off plane insertion of the needle.

11. The apparatus as recited in claim 5, wherein said curved line is a curved edge of said plate.

12. The apparatus as recited in claim 5, further comprising a sheet joined to said bottom and a sheet joined to the top for maintaining a sterile field.

13. The apparatus as recited in claim 5, further comprising a needle holder pivotally joined to said adapter for allowing the needle to pivot in said third plan.

14. The apparatus as recited in claim 12, wherein said needle holder further comprises a needle canal.

15. The apparatus as recited in claim 12, further comprising a joint and a bridge for joining said needle holder to said adapter.

16. An apparatus for a needle director for an ultrasound transducer probe, the apparatus comprising:
an adapter for joining to a distal portion of the ultrasound transducer probe, said adapter comprising a bottom residing in a first plane parallel to a distal end of the joined ultrasound transducer probe and perpendicular to a midline of the ultrasound transducer probe where the midline extends through a center of a central plane of the ultrasound transducer probe;
a needle insertion site disposed in said first plane for allowing the needle to pass below said first plane;

17. The apparatus as recited in claim 16, further comprising:
A needle with at least one marker that is certain distance from the needle tip; and
a plate joined to said adapter for guiding the needle, said plate disposed in a second plane parallel to and offset from a third plane containing the midline and said needle insertion site, said plate comprising a flat edge positioned in said first plane and a first flat surface proximate said third plane for guiding an insertion of the needle along said third plane, said first flat surface comprising a plurality of radial lines corresponding to insertion directions extending through said needle insertion site, each of said plurality of radial lines having an indicator of a distance along the midline where a corresponding insertion direction intersects, said first flat surface further comprising at least one curved line intersecting said plurality of radial lines for aligning with said marker on the needle for indicating that a tip of an inserted needle has intersected the midline at a distance indicated by at least one of said indicators.

18. The apparatus as recited in claim 15, wherein said third plane is in said central plane for an in plane insertion of the needle.

19. The apparatus as recited in claim 15, wherein said third plane is perpendicular to said central plane for a typical off plane insertion of the needle.

20. The apparatus as recited in claim 15, wherein said third plane is any plane that contains said midline in said central plane others than planes recited in claims 15 and 16 for an atypical off plane insertion of the needle.

21. The apparatus as recited in claim 15, wherein said curved line is a curved edge of said plate.

22. The apparatus as recited in claim 15, wherein said adapter surrounds the distal portion of the ultrasound transducer probe.

23. The apparatus as recited in claim 15, further comprising a sheet joined to said bottom and a sheet joined to the top for maintaining a sterile field.

24. The apparatus as recited in claim 15, further comprising a needle holder pivotally joined to said adapter for holding the needle and allowing the needle to pivot in said third plan about said insertion site.

25. The apparatus as recited in claim 22, wherein said needle holder further comprises a needle canal.

26. The apparatus as recited in claim 22, further comprising a joint and a bridge for joining said needle holder to said adapter.
